(19) United States
(12) Patent Application Publication
(10) Pub. No.: US 2010/0120137 A1
(43) Pub. Date: May 13, 2010

(54) Apparatus for growing biological mass

(76) Inventors: Oleg Shvabsky, Hallandale Beach, FL (US); Jacob Gitman, Bay Harbor Island, FL (US)

Correspondence Address: llya Zborovsky, 6 Schoolhouse Way, Dix Hills, NY, 11746, US

(21) Appl. No.: 12/292,191

(22) Filed: Nov. 13, 2008

(51) Int. Cl. C12M 3/06; C12M 3/00

(52) U.S. Cl. 435/297.1; 435/289.1

(57) ABSTRACT

An apparatus for growing biological mass has a container for accommodating and growing a biological mass, a unit for supplying liquid in the container, a unit for supplying CO₂ in the container, a unit for supplying a feedstock in the container, and a cavitating device arranged so that a liquid is supplied from the unit for supplying liquids directly into the cavitating device and CO₂ is supplied to the cavitating device directly from the unit for supplying CO₂, so that in the cavitating device a mixture of liquid and CO₂ is produced with a cavitation process, the cavitating device being connected with the container for supplying the mixed CO₂ and liquid subjected to cavitation into the container.

### BACKGROUND OF THE INVENTION

The present invention relates to apparatuses for growing a biological mass, for example algae for production of biofuels.

It is known to grow a biological mass, in particular algae, and then produce biofuels from them by pressure. In the known apparatuses CO₂ for growing of the biological mass is supplied directly into the container in which the biological mass is accommodated. This however has certain disadvantage that CO₂ is not efficiently used, and also it is difficult to maintain a required level of its concentration in the growing container in which the algae have to grow. Also, it is necessary to provide a significant quantity of CO₂ since it is not completely absorbed by a process, and a part of CO₂ is escaped to atmosphere.

### SUMMARY OF THE INVENTION

Accordingly, it is an object of the present invention to provide an apparatus for growing biological mass, which avoid the disadvantages of the prior art.

In keeping with these objects and with others which will become apparent hereinafter, one feature of the present invention resides, briefly stated, in an apparatus for growing biological mass, comprising a container for accommodating and growing a biological mass; means for supplying liquid in said container; means for supplying CO₂ in said container; means for supplying a feedstock in said container; and a cavitating device arranged so that a liquid is supplied from means for supplying liquids directly into said cavitating device and CO₂ is supplied to said cavitating device directly from said means for supplying CO₂, so that in said cavitating device a mixture of liquid is CO₂ is produced with a cavitation process, said cavitating device being connected with said container for supplying the mixed CO and liquid subjected to cavitator into said container.

Another feature of the present invention resides, briefly stated, in means for preparing a medium arranged so that said cavitating device is connected with said preparing means and said means for supplying feedstock is connected with said preparing means and therefore in said means for preparing a working medium the liquid means is mixed with CO₂ with the cavitation process and supplied to said means for preparing a working medium where it is mixed with the feedstock, and thereafter is supplied from said means for preparing a working medium into said container.

Still a further feature of the present invention resides, briefly stated, in means for withdrawing an excessive liquid from said container into said means for supplying the liquid.

Still a further feature of the present invention resides, briefly stated, in a filter arranged between said container and said means for supplying liquid so that from said container a liquid is supplied into said means for supplying liquid after being filtered.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a side view of an apparatus for growing biological mass; and

FIG. 2 is a plan view of an apparatus for growing biological mass.

### DESCRIPTION OF THE PREFERRED EMBODIMENTS

An apparatus for growing biological mass, for example algae for producing biofuels, includes a container 1 for growing biomass. A biomass is introduced into the container 1 and the algae grow in presence of water and CO₂.

Liquid is supplied in the container 1 from a liquid container 3, a feedstock is supplied into the container from a feedstock container 4, CO₂ is supplied into the container 1 from CO container 5.

In accordance with the present invention the liquid container 3 and the CO₂ container 5 are not directly connected to the container for growing a biomass 1. Instead, a cavitator 7 is provided into which the liquid is supplied from the liquid container 3 and CO₂ is supplied from the CO₂ container 5. In the cavitator 7 the liquid is mixed with the CO₂ under the action of cavitation which is generated in the cavitator 7. The cavitator 7 can be a known cavitating device which cavitation is achieved by, known methods. One of such cavitators can be a device in which excitation is achieved by whirl-generating pipes creating contacts of outer layers of whirling flows, that generates their acoustic excitation by deformation interaction (RF 2268772). In the cavitator 7 a homogenous mixture of CO₂ in liquid is produced on a molecular level. The thusly produced mixture is supplied to the main container 1 for growing algae.

In accordance with a further feature of the present invention, the thusly produced mixture after cavitator is mixed with the feedstock supplied from the feedstock container 4 in a container 6 for preparation of a working medium. In the container 6 the working medium is prepared, which includes cavitated liquid with CO₂ and feedstock. This working medium is supplied then by a pump 8 into the container 1, preferably through a collector 9. The collector 9 is which is a tubular collector extending around the peripheral wall of the container 1 and provided with several pipes communicating the interior of the collector 9 with the interior of the container 1.

In the container 1, as known, the biological mass is grown, for example algae for producing biofuels.

With the apparatus is designed in accordance with the present invention, CO₂ is completely absorbed in water, the biological mass obtains better contact with the CO₂ distributed in water by a cavitation process, a necessary level of concentration of CO₂ is easily maintained by adding it from the cavitator 7, and a process of growing biomass is substantially accelerated.

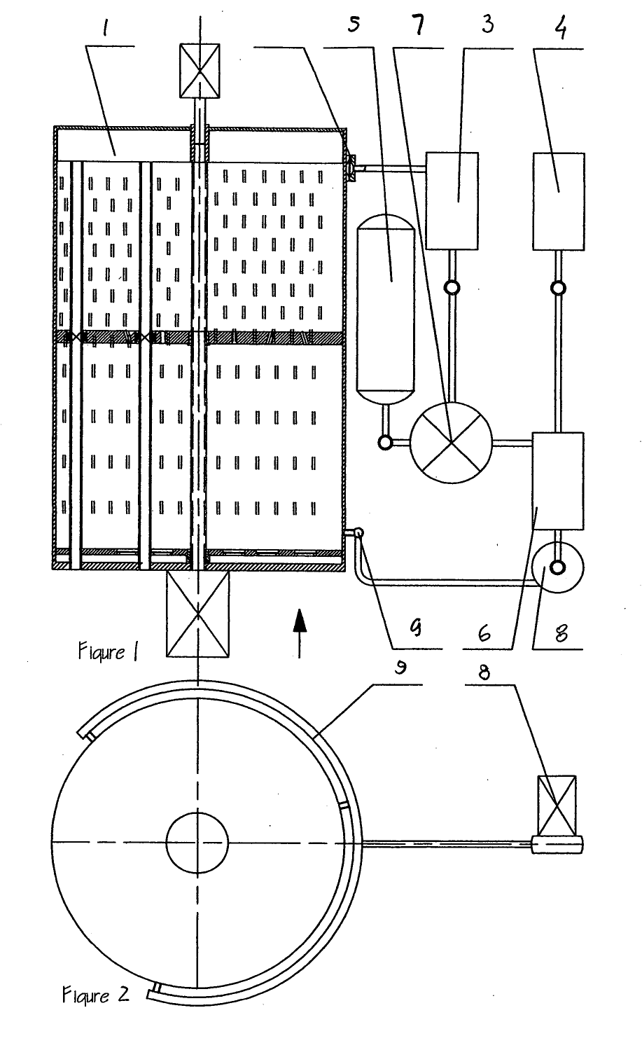

As can be seen from FIG. 1, an excessive liquid is supplied from the container 1 into the liquid container 5 through a pipe with a filter 2 therebetween, which filters out biological mass and allows only the liquid to pass into the liquid container 3.

It will be understood that each of the elements described above, or two or more together, may also find a useful application in other types of constructions differing from the type described above.

While the invention has been illustrated and described as embodied in an apparatus growing biological mass, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. An apparatus for growing biological mass, comprising a container for accommodating and growing a biological mass; means for supplying liquid in said container; means for supplying CO₂ in said container; means for supplying a feedstock in said container; and a cavitating device arranged so that a liquid is supplied from said means for supplying liquid directly into said cavitating device and CO₂ is supplied to said cavitating device directly from said means for supplying CO₂, so that in said cavitating device a mixture of liquid and CO₂ is produced with a cavitation process, said cavitating device being connected with said container for supplying the mixed CO₂ and liquid subjected to cavitation into said container.

2. An apparatus as defined in claim 1, and further comprising means for preparing a medium arranged so that said cavitating device is connected with said preparing means and said means for supplying feedstock is connected with said preparing means and therefore in said means for preparing a working medium the liquid mixed with CO in the cavitation process and supplied to said means for preparing a working medium is mixed with a feedstock, and thereafter is supplied from said means for preparing a working medium into said container.

3. An apparatus as defined in claim 1; and further comprising means for withdrawing an excessive liquid from said container into said means for supplying the liquid.

4. An apparatus as defined in claim 3; and further comprising a filter arranged between said container and said means for supplying liquid so that from said container a liquid is supplied into said means for supplying liquid after being filtered.
